(19) United States
(12) Patent Application Publication
(10) Pub. No.: US 2021/0315789 A1
(43) Pub. Date: Oct. 14, 2021

(54) IRON CHELATORS FOR TREATING AESTHETIC SKIN CONDITIONS

(71) Applicant: TAUTONA GROUP IP HOLDING COMPANY, L.L.C., Redwood City, CA (US)

(72) Inventor: Geoffrey C. GURTNER, Stanford, CA (US)

(73) Assignee: TAUTONA GROUP IP HOLDING COMPANY, L.L.C., Redwood City, CA (US)

(21) Appl. No.: 17/273,855

(22) PCT Filed: Sep. 20, 2019
(86) PCT No.: PCT/US2019/052192

§ 371 Date: Mar. 5, 2021

Related U.S. Application Data
(60) Provisional application No. 62/734,164, filed on Sep. 20, 2018.

(51) Int. Cl. A61K 8/42; A61K 8/11; A61K 8/02; A61Q 19/00; A61Q 19/02; A61Q 19/08; A61K 31/16; A61K 9/00; A61K 9/06; A61K 9/70

(52) U.S. Cl. CPC A61K 8/42; A61K 8/11; A61K 8/0291; A61K 8/0208; A61Q 19/007; A61K 2800/92; A61Q 19/08; A61K 31/16; A61K 9/0014; A61K 9/06; A61K 9/7023; A61Q 19/02

(57) ABSTRACT

Provided herein are iron chelating compounds, compositions containing such compounds and methods of using the compounds in treating various aesthetic skin conditions. The iron chelating compositions may be administered topically or orally. Treating the aesthetic skin conditions includes preventing onset or delaying progression of the aesthetic skin condition.

### CROSS REFERENCE TO RELATED APPLICATION

This application claims benefit of U.S. Provisional Application No. 62/734,164, filed Sep. 20, 2018, which disclosure is incorporated herein by reference in its entirety.

### INCORPORATION BY REFERENCE

All publications and patent applications mentioned in this specification are incorporated herein by reference in their entirety to the same extent as if each individual publication or patent application was specifically and individually indicated to be incorporated by reference.

### BACKGROUND

Aging, chronic exposure to adverse environmental factors, malnutrition, fatigue, etc., can change the visual appearance, physical properties, or physiological functions of skin in ways that are considered visually undesirable. The most notable and obvious changes include the development of fine lines and wrinkles, loss of elasticity, increased sagging, loss of firmness, loss of color evenness or tone, coarse surface texture, and mottled pigmentation. Less obvious, but measurable changes which occur as skin ages or endures chronic environmental insult include a general reduction in cellular and tissue vitality, reduction in cell replication rates, reduced cutaneous blood flow, reduced moisture content, accumulated errors in structure and function, alterations in the normal regulation of common biochemical pathways, and a reduction in the skin's ability to remodel and repair itself. Many of the alterations in appearance and function of the skin are caused by changes in the outer epidermal layer of the skin, while others are caused by changes in the lower dermis. Therefore, there is a need to develop new compounds, methods and compositions for preventing and/or treating skin conditions related to the aesthetic appearance of skin.

### SUMMARY OF THE DISCLOSURE

In a first aspect, a method of treating an aesthetic skin condition in a subject's skin is provided, the method including contacting the subject's skin in need thereof with an effective amount of an iron chelator compound or a pharmaceutically acceptable salt thereof, or a composition including an iron chelator compound or a pharmaceutically acceptable salt thereof, where the aesthetic skin condition is prevented or treated.

In some variations, the iron chelator compound may be selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin. In some embodiments, the iron chelator compound may be deferoxamine, deferiprone or deferasirox. In some embodiments, the iron chelator compound may be deferoxamine. In some variations, the aesthetic skin condition may be a decreased hair density, thinning skin, a wrinkle, a fine line on skin, dry, flaky or itchy skin, alopecia, aging, skin discoloration, a sun spot, an age spot, a dark circle under eyes, bruising following injury or surgery, a liver spot, scar, spider vein, or rosacea.

In some variations, the effective amount of an iron chelator compound or a pharmaceutically acceptable salt thereof, or a composition including an iron chelator compound or a pharmaceutically acceptable salt thereof may be administered topically.

In some variations, the iron chelator compound may be encapsulated in a reverse micelle structure with at least one surfactant. In some other variations, the iron chelator compound may not be encapsulated in a reverse micelle.

In some variations, contacting the subject's skin with the composition including the iron chelator compound may further include releasing the iron chelator compound from the composition over a treatment period, and penetrating the iron chelator compound into the skin in need of treatment. In some embodiments, releasing the iron chelator compound may further include releasing the iron chelator compound from within a matrix of the composition. In some embodiments, the matrix may include ethylcellulose.

In some variations, the composition to be applied topically may further include polyvinylpyrrolidone (PVP).

In some variations, the iron chelator compound has a concentration of at least about 0.1% and not more than about 40% as weight/weight percent of the composition. In some embodiments, the iron chelator compound may have a concentration of at least 10% w/w % or about 15% w/w.

In some variations, the composition may be a cream or a lotion.

In some variations, the composition may be a film. In some embodiments, applying the compositions may include applying to the skin a transdermal patch containing the composition.

In some variations, the effective amount of an iron chelator compound or a pharmaceutically acceptable salt thereof, or a composition including an iron chelator compound or a pharmaceutically acceptable salt thereof may be administered orally.

In another aspect, a formulation is provided comprising an iron chelator compound or a pharmaceutically acceptable salt thereof in a cream, lotion or film composition formulated for transdermal aesthetic skin treatment. In some variations, the iron chelator compound may be stabilized in the cream, lotion or film composition. In some variations, the iron chelator compound may be encapsulated in a reverse micelle structure.

In some variations, the iron chelator compound or pharmaceutically acceptable salt thereof may be encapsulated with at least one surfactant within the reverse micelle.

In some variations, the at least one surfactant may include one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols; and CTAB (cetyl trimethylammonium bromide). In some embodiments, the at least one surfactant includes at least one of Polysorbate 80 and sorbitan monolaurate (Span20). The at least one surfactant may be present at a concentration of 1% w/w to 25% w/w.

In some variations, the reverse micelles may further include polyvinylpyrrolidone. The polyvinylpyrrolidone may be present at a concentration of 0.1% to 25% w/w.

In some variations, the iron-chelating compound may be present within the formulation in a concentration from 1% to 35% w/w. In some embodiments, the iron-chelating compound is present within the formulation at a concentration of 13% w/w.

In some variations, the formulation may be a lotion or cream.

In some variations, the formulation may further include a matrix. In some embodiments, the matrix may be a biodegradable polymer. In some embodiments, the biodegradable polymer may include ethyl cellulose. In some embodiments, ethyl cellulose may be present at a concentration from 25% w/w to 75% w/w.

In some variations, the formulation of the iron-chelating compound may be a film or a patch.

In some variations, the formulation may be configured to be compatible with facial skin or a scalp of a subject.

In another aspect, a method of treating a skin condition associated with oxidation of skin cells is provided, the method including topically applying to skin in need thereof an iron chelator compound or a pharmaceutically acceptable salt thereof, or a composition including an iron chelator compound, or a pharmaceutically acceptable salt thereof, where topical application of the compound, salt or composition treats oxidation of skin cells.

In some variations, applying may include applying to the skin a transdermal patch containing the composition.

In some variations, the iron chelator compound may be selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin. In some embodiments, the iron chelator compound may be DFO.

In some embodiments, the iron chelator compound may be encapsulated in a reverse micelle structure with a surfactant. In some embodiments, the iron chelator compound may be encapsulated within the reverse micelle structure within a matrix. In some embodiments, the matrix mayinclude a biodegradable polymer.

In some variations, the composition may further include polyvinylpyrrolidone (PVP).

In another aspect, a method for preventing and/or treating an aesthetic skin condition in a subject's skin is provided, the method including: contacting the subject's skin in need thereof with an effective amount of an iron chelator compound or a pharmaceutically acceptable salt thereof, or a composition including an iron chelator compound or a pharmaceutically acceptable salt thereof, where the aesthetic skin condition is prevented or treated. In some variations, the aesthetic skin condition may be a decreased hair density, thinning skin, a wrinkle, a fine line on skin, alopecia, aging, skin discoloration, a sun spot, an age spot, a dark circle under eyes, bruising following injury or surgery, a liver spot, scar, spider vein, or rosacea. In some variations, the iron chelator compound may have a concentration of at least about 0.1% and not more than about 40% as weight/weight percent of the composition.

In another aspect, a method for improving, preventing and/or treating an aesthetic skin condition in a subject is provided, the method including: administering to the subject in need thereof an effective amount of an iron chelator compound, or a pharmaceutically acceptable salt thereof, or a composition containing an iron chelator compound or a pharmaceutically acceptable salt thereof, thereby improving and/or treating the aesthetic skin condition.

In another aspect a method for preventing and/or treating an aesthetic skin condition in a subject's skin is provided, the method including: contacting the subject's skin in need thereof with a transdermal patch including a film including an iron chelator compound or a pharmaceutically acceptable salt thereof encapsulated in a reverse micelle structure with surfactant within a matrix, where the encapsulated iron chelator compound is released from the matrix over a treatment period, and penetrated into the skin.

In another aspect, a method of lightening skin or evening skin tone is provided, the method including topically applying to skin in need thereof an iron chelator compound or a pharmaceutically acceptable salt thereof, or a composition including an iron chelator compound, where topical application of the compound, salt or composition lightens skin or evens skin tone.

In another aspect, a method of reducing the appearance of symptoms associated with erythema is provided, the method including topically applying to skin in need thereof an iron chelator compound, or a pharmaceutically acceptable salt thereof, or a composition including an iron chelator compound, or a pharmaceutically acceptable salt thereof, where topical application of the compound, salt or composition reduces the appearance of symptoms associated with erythema.

In another aspect, a method of treating dry, flaky, or itchy skin or reducing the appearance of uneven skin tone is provided, the method including topically applying to skin in need thereof an iron chelator compound or a pharmaceutically acceptable salt thereof, or a composition including an iron chelator compound or a pharmaceutically acceptable salt thereof, where topical application of the compound, salt or composition treats dry, flaky, or itchy skin or reducing the appearance of the uneven skin.

In another aspect, a method of reducing the appearance of fine lines or wrinkles of skin is provided, the method including topically applying to the skin in need thereof an iron chelator compound, or a pharmaceutically acceptable salt thereof, or a composition including an iron chelator compound, or a pharmaceutically acceptable salt thereof, where topical application of the composition reduces the fine lines or wrinkles of the skin.

In another aspect, a method of treating hyperpigmentation of skin is provided, the method including topically applying to the skin in need thereof an iron chelator compound, or a pharmaceutically acceptable salt thereof, or a composition including an iron chelator compound or a pharmaceutically acceptable salt thereof, where topical application of the compound, salt, or composition treats hyperpigmentation of the skin.

In another aspect, a method of thickening hair or treating or preventing hair loss on the scalp is provided, the method including topically applying to the skin in need thereof an iron chelator compound, or a pharmaceutically acceptable salt thereof, or a composition including an iron chelator compound or a pharmaceutically acceptable salt thereof, where topical application of the compound, salt or composition thickens hair or treats or prevents hair loss on the scalp.

In another aspect, a kit for treating aesthetic skin conditions is provided, including a composition comprising an iron chelator compound; and directions for its use.

### DETAILED DESCRIPTION

### Definitions

The terms “treating”, and “treatment” and the like are used herein to generally mean

one or more of (1) inhibiting the disease; e.g., inhibiting an aesthetic skin disease, condition or disorder in an individual who is experiencing or displaying the pathology or symptomatology of the disease, condition or disorder (i.e., arresting further development of the pathology and/or symptomatology); and (2) ameliorating the aesthetic skin disease or condition; e.g., ameliorating a skin disease, condition or disorder in an individual who is experiencing or displaying the pathology or symptomatology of the aesthetic skin disease, condition or disorder (i.e., reversing the pathology and/or symptomatology) such as decreasing the severity of disease. The compounds and/or compositions and/or devices and methods of the present disclosure are useful in preventing or reducing the risk of developing any of the aesthetic skin diseases referred to herein; e.g., preventing, improving or reducing the risk of developing an aesthetic skin disease, condition or disorder in an individual who may be predisposed to the aesthetic skin disease, condition or disorder but does not yet experience or display the pathology or symptomatology of the disease. Thus “treating” encompasses preventing an aesthetic skin condition.

The terms “prevent”, “preventing”, “prevention” and grammatical variations thereof as used herein, means a method of partially or completely delaying or precluding the onset or recurrence of a disease, disorder or condition and/or one or more of its attendant symptoms or barring a subject from acquiring or reacquiring a disorder or condition or reducing a subject's risk of acquiring or reacquiring a disorder or condition or one or more of its attendant symptoms.

The term “administering” means oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “composition” or “pharmaceutical composition” means a formulation suitable for administration to an intended animal subject for therapeutic purposes that contains at least one pharmaceutically active compound and at least one pharmaceutically acceptable carrier or excipient.

The term “subject” includes mammals, e.g. cats, dogs, horses, pigs, cows, sheep, rodents, rabbits, squirrels, bears, primates such as chimpanzees, gorillas, and humans which may suffer from aesthetic skin conditions.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

“Pharmaceutically acceptable salts” means salt compositions that is generally considered to have the desired pharmacological activity, is considered to be safe, non-toxic and is acceptable for veterinary and human pharmaceutical applications. Such salts include acid addition salts formed with inorganic acids such as hydrochloric acid, hydrobromic acid, sulfuric acid, phosphoric acid, and the like; or with organic acids such as acetic acid, propionic acid, hexanoic acid, malonic acid, succinic acid, malic acid, citric acid, gluconic acid, salicylic acid and the like.

The term “topical application” or “applying topically” means to apply or spread a composition onto the surface of tissue. “Topical skin composition” includes compositions suitable for topical application on epidermal and/or dermal cells. Such compositions are typically dermatologically-acceptable in that they do not have undue toxicity, incompatibility, instability, allergic response, and the like, when applied to skin. Topical skin care compositions of the present invention can have a selected viscosity to avoid significant dripping or pooling after application to skin.

The term “effective,” as that term is used in the specification and/or claims, means adequate to accomplish a desired, expected, or intended result.

The term “inhibiting” or “reducing” or any variation of these terms, when used in the claims and/or the specification includes any measurable decrease or complete inhibition to achieve a desired result.

In some embodiments, the disclosure provides a method for improving and/or treating an aesthetic skin condition in a subject's skin. The method includes contacting the subject's skin in need thereof with an effective amount of an iron chelator compound, thereby improving and/or treating the aesthetic skin condition.

The term “aesthetic skin condition” means a skin disease or condition that affects the cosmetic appearance of skin. Exemplary aesthetic skin conditions include, but are not limited to, scars, skin laxity, wrinkles, moles, liver spots, excess fat, cellulite, unwanted hair, skin discoloration, spider veins, dry, flaky, or itchy skin, uneven skin tone, fine lines or wrinkles, inflamed or erythemic skin, oxidative damage, skin having dark spots, sun spot, age spot, melasma, hyperpigmentation, hair loss on the scalp, male-pattern baldness, female-pattern baldness, cicatricial alopecia, alopecia areata telogen effluvium, traction alopecia, anagen effluvium, hair loss on eyebrows, hair loss on eyelashes, rosacea, and the like

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts

### Method of Treating Aesthetic Skin Conditions

Skin is a complex organ of the body and its structure can indicate points of intervention to prevent or ameliorate skin conditions using the compositions of the disclosure. Epidermis is the outermost waterproof protective layer of skin and provides a barrier to infection. The epidermis contains no blood vessels, and cells in the deepest layers are nourished mostly by diffused oxygen from the external atmosphere and to a smaller degree by blood capillaries extending to the outer layers of the dermis. The epidermis can be further subdivided into the following strata (beginning with the outermost layer): corneum, lucidum (only in palms of hands and bottoms of feet), granulosum, spinosum, and basale. The main type of cells which make up the epidermis are keratinocytes (located throughout the strata, Merkel cells and melanocytes which are mainly located within the basale stratum), and Langerhans cells (located throughout the strata). New cells are formed at the innermost layer, and daughter cells move toward the outer strata changing shape and composition as they die due to isolation from their blood source. Nuclear structures and cytoplasmic organelles disappear, cytoplasm is released and keratin protein is produced, retained and polymerized into keratin filaments along with release of cytoplasm. They eventually reach the corneum and slough off. This keratinized layer of skin is responsible for keeping water in the body and for making skin a natural barrier to infection and exogenous materials. The outermost layer of the epidermis includes 25 to 30 layers of dead cells.

The dermis is the layer of skin beneath the epidermis which includes connective tissue and cushions the body from stress and strain. The dermis is tightly connected to the epidermis by a basement membrane.

The dermis is itself divided into two areas: a superficial area adjacent to the epidermis, called the papillary region, and a deep thicker area known as the reticular region. The papillary region is composed of loose areolar connective tissue having papillae projections provide the dermis with a “bumpy” surface that interdigitates with the epidermis, strengthening the connection between the two layers of skin.

The thicker reticular region is composed of dense irregular connective tissue, and receives its name from the dense concentration of collagenous, elastic and reticular fibers that weave throughout it, providing strength, extensibility, and elasticity. Also located within the reticular region are hair follicles containing the roots of hairs, sebaceous glands, sweat glands cutaneous sensory receptors for touch, temperature, and pain, nail bed, lymphatic vessels and blood vessels. The blood vessels in the dermis provide nourishment and waste removal from its own cells as well as from the Stratum basale of the epidermis.

As can be seen from the complex structure and multitude of functions performed within the skin (e.g., elastic, protective, supportive, sensory, amongst many others) by the differing cell types resident in the respective strata, disablement or damage to skin cells (e.g., any cell as described above populating the epidermis or dermis) can lead to impaired function and impaired aesthetics of the skin overall.

Exposure to UV light, age, irradiation, chronic sun exposure, environmental pollutants, air pollution, wind, cold, heat, chemicals, disease pathologies, smoking, or lack of nutrition can contribute to dysregulation or dysfunction of the myriad of growth, repair and replacement processes performed by cells within the epidermis or dermis. A variety of intracellular mechanisms are adversely affected by increased concentration of oxidative species. Alternatively, in some other intracellular mechanisms, other declining rates of growth, repair and/or replacement resulting, for example, from age, may be adversely affected even by normative levels of oxidative species.

It has been reported that use of iron chelator molecules can ameliorate some of these functional defects, and permit better wound healing, for example, in aged populations. (See Bonham et al., Wound Repair Regen 2018 May; 26(3): 300-305. doi: 10.1111/wrr.12667. Epub 2018 Oct. 25) Without wishing to be bound by theory, Applicant have discovered that iron chelator molecule therapy can to extended to use in treating skin conditions subject to the stressors listed above, including oxidative stressors. Reactive oxygen species may be increased by some of the stressors, and the ability of iron chelators to ameliorate the damage at the cellular and tissue level to improve the aesthetic condition of the skin.

Non-limiting examples of skin conditions that can be treated and/or prevented and/or improved with the iron chelating compounds as described herein or a pharmaceutically acceptable salt thereof, or compositions containing the iron chelating compound or a pharmaceutically acceptable salt thereof, include dry skin, itchy skin, flaky skin, inflamed skin, thinning skin, erythemic skin, pain associated with erythemic skin, sensitive skin, pruritus, spider veins, lentigo, age spots, senile purpura, keratosis, melasma, blotches, fine lines or wrinkles, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, pustules, nodules, whiteheads, blackheads, impetigo, erysipelas, erythrasma, eczema, sun burns, burned skin, open wounds, skin-inflammatory skin conditions, etc. In certain non-limiting situations, the skin condition can be caused by exposure to UV light, age, irradiation, chronic sun exposure, environmental pollutants, air pollution, wind, cold, heat, chemicals, disease pathologies, smoking, or lack of nutrition. The skin can be facial skin or non-facial skin (e.g., arms, legs, hands, chest, back, feet, etc.). The method can further comprise identifying a person in need of skin treatment. The person can be a male or female. The age of the person can be at least 1, 2, 3, 4, 5, 6, 7, 8, 9, 10, 15, 20, 25, 30, 35, 40, 45, 50, 55, 60, 65, 70, 75, 80, 85, 90, 95, or more years old, or any range derivable therein. The method can also include topically applying an amount effective to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, Langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin).

In some embodiments, the aesthetic skin conditions treatable with the iron chelators as described herein include, but are not limited to, decreased hair density, wrinkle, alopecia, aging, skin discoloration, sun spots, aging spots, dark circle under eyes, bruising following injury or surgery, liver spots, scar, spider vein, or rosacea.

In some embodiments, the iron chelator compound is encapsulated in a reverse micelle with a non-ionic surfactant within a matrix. The encapsulated iron chelator compound can be released from the matrix over a treatment period, and penetrated into the skin in need of treatment. The release of the iron chelators can be immediate release or sustained release, which may include an extended release profile.

In some embodiments, the iron chelator compound is formulated into a composition suitable for transdermal delivery. In other embodiments, the iron chelator compound is formulated into a film. For transdermal delivery, a patch can be used, wherein the patch contains the iron chelator compound. In some embodiments, the patch includes a film, which contains an iron chelator compound.

In some embodiments, the iron chelator compound is formulated into a composition suitable for topical delivery. In other embodiments, the composition can be a cream, gel or lotion.

In some embodiments, the iron chelator compound used for treating or improving aesthetic skin conditions include, but are not limited to deferoxamine (DFO), deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

In some embodiments, the iron chelator compound is deferoxamine, deferiprone, or deferasirox. In some other embodiments, the iron chelator compound is deferoxamine.

In some embodiments, the iron chelator compound has a concentration of at least about 0.1% and not more than about 20% as weight/weight percent of the composition.

In some embodiments, the concentration of one or more of the iron chelators in the compositions described herein is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more of the iron chelators is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more of the iron chelators is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v.

In some embodiments, the concentration of one or more of the iron chelators is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

The iron chelators described herein are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the iron chelator is administered, the subject to be treated, the body weight of the subject to be treated, and the preference and experience of the attending physician.

A composition described herein typically contains an active ingredient (e.g., an iron chelator or a pharmaceutically acceptable salt and/or coordination complex thereof, and one or more pharmaceutically acceptable excipients, carriers, including but not limited inert solid diluents and fillers, diluents, sterile aqueous solution and various organic solvents, permeation enhancers, solubilizers and adjuvants.

In some embodiments, the disclosure provides a method for improving, preventing and/or treating an aesthetic skin condition in a subject. The method includes administering to the subject in need thereof an effective amount of an iron chelator compound, thereby improving and/or treating the aesthetic skin condition. The iron chelator compound can be formulated into a composition suitable for oral administration.

In some embodiments, the disclosure provides a method for improving, preventing and/or treating an aesthetic skin condition in a subject's skin. The method may include contacting the subject's skin in need thereof with a transdermal patch comprising a film comprising an iron chelator compound encapsulated in a reverse micelle with a non-ionic surfactant within a matrix, wherein the encapsulated iron chelator compound is released from the matrix over a treatment period, and penetrated into the skin.

In some other embodiments, the method may include contacting the subject's skin with a composition including the iron chelator in a suitable formulation, e.g., a lotion, cream or a patch comprising a film including the iron chelator compound where the iron chelator compound is not present within a matrix. In some other embodiments, the method may include contacting the subject's skin with a composition including the iron chelator compound where the iron chelator compound is present in a composition that does not contain a reverse micelle, but may optionally have a stabilizer present, such as for example, polyvinylpyrrolidone (PVP). For example, deferoxamine, which is more hydrophilic than others of the group of iron chelator compounds, may be more effective when administered encapsulated within a reverse micelle, but lotion or cream formulations may, in some embodiments, not contain reverse micelles. Some other iron chelator compounds may be more hydrophobic, such as pyridoxal isonicotinoyl hydrazine (PIH), and may not require encapsulation within reverse micelles but may penetrate the skin when formulated without such encapsulation.

The treatment time can be from 10 minutes to 16 weeks. In some embodiments, the treatment time may be for no longer than 1 day, 5 days, a week, or a month.

In some variations, a composition including an iron chelator compound may be applied to a treatment area in conjunction with technology such as negative pressure wound therapy to enhance penetration to a treatment area. In some embodiment, the composition may include a lotion or a solution formulation of the iron chelator compound.

In some embodiments, the matrix used may include polyvinylpyrrolidone (PVP) and ethylcellulose.

In some embodiments, the disclosure provides a method of lightening skin or evening skin tone. The method includes topically applying to skin in need thereof an iron chelator compound as disclosed herein or a pharmaceutically acceptable salt thereof, or a composition comprising an iron chelator compound or a pharmaceutically acceptable salt thereof, wherein topical application of the compound, salt or composition lightens skin or evens skin tone. The composition can include a skin lightening agent such as hydroquinone.

In some embodiments, the disclosure provides a method of treating a skin condition associated with oxidation of skin cells. The method includes topically applying to skin in need thereof an iron chelator compound as disclosed herein or a pharmaceutically acceptable salt thereof, or a composition comprising an iron chelator compound or a pharmaceutically acceptable salt thereof, wherein topical application of the compound, salt, or composition treats oxidation of skin cells.

In some embodiments, the disclosure provides a method of reducing the appearance of symptoms associated with erythema (e.g., erythemic skin, sensitive skin, inflamed skin) comprising topically applying an iron chelator compound as disclosed herein or a pharmaceutically acceptable salt thereof, or a composition containing an iron chelating compound or a pharmaceutically acceptable salt thereof to skin in need thereof. Erythema can be caused by skin sunburn, electrical treatments of skin, skin burns, contact allergies, systemic allergies, skin toxicity, exercise, insect stings, bacterial infection, viral infection, fungal infection, protozoa infection, massage, windburn, etc.

In some embodiments, the disclosure provides a method of treating dry, flaky, or itchy skin or reducing the appearance of uneven skin tone comprising topically applying an iron chelator compound as disclosed herein or a pharmaceutically acceptable salt thereof, or a composition comprising an iron chelator compound or a pharmaceutically acceptable salt thereof to dry, flaky, or itchy skin or to skin having an uneven skin tone.

In some embodiments, the disclosure provides a method of reducing the appearance of fine lines or wrinkles comprising topically applying to skin having fine lines or wrinkles an iron chelator compound as disclosed herein or a pharmaceutically acceptable salt thereof, or a composition comprising an iron chelator compound or a pharmaceutically acceptable salt thereof.

In certain embodiments, the compositions containing the iron chelating compound can decrease the amount of internal oxidation and/or external oxidative damage in a cell. In other embodiments, the compositions can increase collagen synthesis in a cell. The compositions can also reduce skin inflammation, such as by reducing inflammatory cytokine production in a cell. Non-limiting examples of such cells include human epidermal keratinocyte, human fibroblast dermal cell, human melanocytes, three dimensional human cell-derived in vitro tissue equivalents comprising human keratinocytes, human fibroblasts, or human melanocytes, or any combination thereof (e.g., combination of human keratinocytes and human fibroblasts or a combination of human keratinocytes and human melanocytes).

In some embodiments, the disclosure provides a method of treating hyperpigmentation comprising applying an iron chelator compound as disclosed herein or a pharmaceutically acceptable salt thereof, or a composition comprising an iron chelator compound or a pharmaceutically acceptable salt thereof to the skin. The method can also comprise identifying a person in need of treating hyperpigmentation and applying an iron chelator compound as disclosed herein or a pharmaceutically acceptable salt thereof, or a composition comprising an iron chelator compound or a pharmaceutically acceptable salt thereof to a portion of the skin exhibiting hyperpigmentation. Additional methods include methods for reducing the appearance of an age spot, a skin discoloration, or a freckle, reducing or preventing the appearance of fine lines or wrinkles in skin, or increasing the firmness of skin by applying an iron chelator compound as disclosed herein or a pharmaceutically acceptable salt thereof, or a composition comprising an iron chelator compound or a pharmaceutically acceptable salt thereof to skin in need of such treatment.

In some embodiments, the disclosure provides a method of thickening hair or treating or preventing hair loss on the scalp (e.g., male-pattern baldness, female-pattern baldness, cicatricial alopecia, alopecia areata telogen effluvium, traction alopecia, anagen effluvium), eyebrows, or eyelashes comprising administering to a patient in need of any such treatment an iron chelator compound as disclosed herein or a pharmaceutically acceptable salt thereof, or a composition comprising an iron chelator compound or a pharmaceutically acceptable salt thereof. The method can also include combining any one of the compositions with known hair loss or hair thickening treatments (e.g., 5-alpha. reductase inhibitors (e.g., finasteride, dutasteride, saw palmetto extract etc.), vasodilators (e.g., minoxidil), ketoconazole, hair transplantation procedures, hair multiplication procedures, laser therapy, caffeine, etc.).

The compositions disclosed herein can also take the form of topically spreadable compositions, sprayable compositions, aerosolized compositions, injectable compositions, edible compositions, compositions in tablet, gel cap, or pill form.

Kits that include the compositions containing iron chelating compounds are also contemplated. In certain embodiments, the composition is comprised in a container. The container can be a bottle, dispenser, or package. The container can dispense a pre-determined amount of the composition. In certain aspects, the compositions is dispensed in a spray, dollop, or liquid. The container can include indicia on its surface. The indicia can be a word, an abbreviation, a picture, or a symbol.

In some embodiments, the iron chelator composition can be formulated as topical skin composition. The composition can have a dermatologically acceptable vehicle or carrier for the iron chelator compound. The composition can further include a moisturizing agent or a humectant, emollient, a surfactant, a silicone containing compounds, a UV agent, an oil, and/or other ingredients known in the art. As the composition may be applied to the face, neck, scalp, arms or other skin areas that are not callused or horny skin, moisturizing components may be particularly useful. The composition can be a lotion, cream, gel, serum, emulsion (e.g., oil-in-water, water-in-oil, silicone-in-water, water-in-silicone, water-in-oil-in-water, oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, etc. The composition can be in powdered form (e.g., dried, lyophilized, particulate, etc.). The composition can be formulated for topical skin application at least 1, 2, 3, 4, 5, 6, 7, or more times a day during use. In some embodiments, the compositions containing the iron chelating compound can be storage stable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein.

In some embodiments, the iron chelator composition can have a pH of about 6 to about 9. In other aspects, the pH can be 1, 2, 3, 4, 5, 6, 7, 8, 9, 10, 11, 12, 13, or 14.

The iron chelator compositions can also include any one of, any combination of, or all of the following additional ingredients: water, a moisturizing agent, a preservative, a thickening agent, a silicone containing compound, an essential oil, a structuring agent, a vitamin, a pharmaceutical ingredient, or an antioxidant, or any combination of such ingredients or mixtures of such ingredients. In certain embodiments, the composition can include at least two, three, four, five, six, seven, eight, nine, ten, or all of these additional ingredients identified in the previous sentence. The amounts of such ingredients can range from 0.0001% to 99.9% by weight or volume of the composition, or any integer or range in between.

### Composition for Oral Administration

In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions described herein to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the composition.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide, N-methyl pyrrolidones, monooctanoin, diethylene glycol monoethyl ether, and water.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Compositions for Topical (e.g., Transdermal) Delivery

Any of the iron chelator molecules, such as deferoxamine, deferiprone, or deferasirox may be formulated in a patch, thin film, gel or lotion. In some variations, the iron chelator may be deferoxamine and may be formulated as a patch. In some variations, the iron chelator may be deferoxamine and may be formulated as a thin film. In some variations, the iron chelator may be deferoxamine and may be formulated as a gel or lotion. In some variations, the iron chelator molecule, such as deferoxamine, deferiprone, or deferasirox, may be present at a concentration of at least about 1%, about 2%, about 3%, about 5% about 7.5%, about 12%, about 15%, about 18%, and not more than about 35%; not more than about 50%; not more than about 75%; not more than about 20%; not more than about 15%, not more than about 12.5% w/w; or any number between the enumerated concentrations. In some variations, the iron chelator molecule may be present at a concentration of about 15%, as weight/weight percent of iron chelator molecule: formulation components within the lotion, gel, film or patch. Depending on the particular iron chelator utilized, the concentration of iron chelator may be higher or lower for effective iron chelation as different iron chelators chelate iron in a 1:1; 1: 2; 1:3 or 3:1; 2:1 ratio of chelator: iron moiety.

Compositions described herein can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Reverse Micelles. The iron-chelating molecule formulations, including extended release formulations of the iron-chelating compound, may include reverse micelles including the iron-chelating compound. Reverse micelles may be dispersed in a biodegradable polymer, e.g., a matrix, such as ethyl cellulose, and form a film, or may be dispersed in a lotion or gel vehicle as described herein. Upon dissolution of the biodegradable polymer, the reverse micelles enter the stratum corneum and disintegrate. PVP dissolves and the iron-chelating molecule is delivered to the dermis. Specifically, the iron-chelating molecule migrates from the extended release transdermal delivery system to the skin following application. Once through the hydrophobic stratum corneum, the reverse micelles can then disintegrate in the more hydrophilic, aqueous environment of the dermis. Thus, a controlled release over a predictable time period may be achieved.

Extended Release Properties. In some variations, the extended release formulation may release the iron chelator compound over a period of about 4 hr, about 8 hr, about 12 hr, about 24 hr, about 48 hr or more. In some variations, the extended release formulation may be a controlled release formulation where the iron chelator is released in a predetermined pattern over a period of time, which may be about 2 hr, about 4 hr, about 8 hr, about 12 hr, about 24 hr, about 48 hr or more.

The reverse micelles may include a non-ionic surfactant. The nonionic surfactant can also provide for formation of reverse micelles, which advantageously aid in delivery of the iron chelator. Suitable surfactants for this purpose can include TWEEN 85® (Polyoxyethylene (20) Sorbitan Trioleate); phospholipids, e.g. lecithin; fatty acid esters such as Plurol® Oleique CC 497 (Gattefosse, Triglyceryl Monooleate); TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols such as cetyl alcohol; and CTAB (cetyl trimethylammonium bromide). Fatty acid esters are long chain aliphatic carboxylic acid, 4 carbons to 26 carbons in length, which are esterified with alcohols, which may include aliphatic alcohols or glycerols. Fatty alcohols are long chain primary alcohols generally having from about 4 carbons to about 26 carbons and are generally straight chain alcohols such as lauryl, strearyl, oleyl, and cetyl alcohols. In some variations, the reverse micelles may include at least one nonionic surfactant, which may be Polysorbate 80 and/or sorbitan monolaurate (Span20). In other variations, the reverse micelles may include at least one nonionic surfactant, which may be Triglyceryl monooleate. In yet other variations, the reverse micelles may include at least one of a fatty acid ester (e.g., Triglyceryl monooleate (Plurol® Oleique)) and a fatty alcohol (e.g., cetyl alcohol). The surfactant may be present at a concentration of from about 0.1 wt % to about 25 weight%, about 10 wt % to about 20 wt %, about 12 wt % to about 18 wt %, about 14 wt % to about 17 wt %, about 15 wt %, about 16 wt %, about 17 wt %, or any value between any of these specifically cited values. If more than one surfactant is included, such as two surfactants, the two surfactants may be present in about 1:10; about 1:8: about 1:6: about 1:4; about 1:2; or about 1:1 w/w ratio to each other. Other useful non-ionic surfactants include Pluronics® block copolymers of poly(ethylene oxide) (PEO) and poly(propylene oxide) (PPO), which have an amphiphilic character with useful properties for the formulations described herein.

Stabilizer. Owing to its hydrophilicity and tendency to crystallize, iron chelator compounds such as, but not limited to, DFO, are especially well suited for delivery when complexed with Polyvinylpyrrolidone (PVP). PVP is known to stabilize drugs in an amorphous form and to promote permeation of hydrophilic molecules. PVP may also be included in the extended release formulation to stabilize the iron chelator molecule within the reverse micelles. For example, PVP may be present at a concentration of from about 0.1 w/w % to about 25 w/w %, about 7 w/w % to about 20 w/w %, about 8 w/w % to about 18 w/wt %, about 10 w/w % to about 16 w/w %, about 10 w/w %, about 12 w/w %, about 14 w/w %, about 16 w/w %.

Other molecules for prevention of crystallization. The formulations may alternatively or additionally comprise other molecules to prevent crystallization of the iron chelator within the formulation, which may prevent crystallization within a vehicle or within the reverse micelles within a vehicle such as a lotion, gel, film or patch. They may include surfactants, emollients, fatty alcohols, fatty esters and the like. Such additives include, without limitation, one or more additives selected from octyldodecanol at a concentration of from about 1.5 to about 4% w/w of polymer; dextrin derivatives at a concentration of from about 2% to about 5% w/w of polymer; polyethylene glycol (PEG) at a concentration of from about 2% to about 5% w/w of polymer; polypropylene glycol (PPG) at a concentration of from about 2% to about 5% w/w of polymer; mannitol at a concentration of from about 2% to about 4% w/w of polymer; Poloxamer 407, 188, 401 and 402 at a concentration of from about 5% to about 10% w/w of polymer; and Poloxamines 904 and 908 at a concentration of from about 2% to about 6% w/w of polymer. These compounds may also aid in penetration of the iron chelator into the skin.

Matrix and optional backing. Whether the formulation is a patch, thin film, gel or lotion, it may include an extended release formulation including a matrix, which may be a biodegradable polymer. The biodegradable polymer may include a natural polymer, a synthetic polymer, or a combination of a natural polymer and a synthetic polymer. Suitable biodegradable polymers useful in the formulation include hydrophilic gelling agents, including but not limited to, carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose (i.e., sodium carboxymethylcellulose 7H 4F) and polyethylene.

The matrix, e.g., a biodegradable polymer, may be present in the lotion, gel, film or patch in a concentration from about 25% w/w to about 75% w/w, about 35% w/w to about 65% w/w, about 40% w/w to about 60% w/w, about 45% w/w to about 55% w/w, about 45% w/w, about 48% w/w, about 50% w/w, about 51% w/w, about 52% w/w, about 53% w/w, about 54% w/w, or about 55% w/w. In some embodiments, the matrix may be present in the film or path in a concentration from about 40% w/w to about 60% w/w, or about 50% w/w to about 55% w/w. In some embodiments, a lotion or cream may not include a matrix, but may include any other suitable vehicle or components as described herein.

Permeability enhancer. In some embodiments, the formulation may include a permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP), which may be provided at a weight/weight concentration of from about 0.1% to about 10%, from about 2.5% to about 7.5%, or about 5%.

Cream or lotion formulations. In some variations, the iron chelator molecule may be formulated in a gel or lotion composition (e.g., formulation). The iron chelator formulation may include reverse micelles and the extended release components of the formulation as described herein, and may include any of the additional components as described herein such as stabilizers, permeation enhancers, crystallization inhibitors, and the like. The iron chelator lotion or cream formulation may include a pharmaceutically acceptable vehicle to act as a diluent, dispersant or carrier, so as to facilitate its distribution and uptake when the formulation is applied to the skin. Vehicles other than or in addition to water can include liquid or solid emollients, solvents, humectants, thickeners and powders.

For topical delivery, an iron chelator molecule, including but not limited to, deferoxamine embedded within a poloxamer gel (Pluronic® F127) provides an efficient and targeted means of delivery. Hydrogels responsive to external stimuli such as pH or temperature may be included. Hydrogels are based on different polysaccharides, such as alginate, cellulose, chitosan, and dextran, which in turn respond to different environmental stimuli. Specifically, a chitosan based hydrogel can be manipulated to respond to temperature and pH in various applications. Likewise, poloxamers such as P188 can be employed as a drug delivery gel and has demonstrated cytoprotective effects in animal models.

The pharmaceutically acceptable vehicle may be present in 5% to 99.9%, preferably from 25% to 80% by weight of the composition, and can, in the absence of other adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the iron-chelating molecules are formulated in an emulsion, the proportion of the fatty phase may be from about 5% to about 80% by weight, and preferably from about 5% to about 50% by weight, relative to the total weight of the composition. Oils, emulsifiers and co-emulsifiers incorporated in the composition in emulsion form are selected from among those used conventionally in the cosmetic or dermatological field. The emulsifier and emulsifier may be present in the composition at a proportion from about 0.3% to about 30% by weight, or about 0.5% to about 20% by weight, relative to the total weight of the composition.

When the iron-chelating molecules are formulated as an oily solution or gel, the fatty phase may constitute more than about 90% of the total weight of the composition. Exemplary oils which may be used according to this invention include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Exemplary hydrocarbons which may serve as emollients are those having hydrocarbon chains anywhere from about 12 to about 30 carbon atoms. Specific examples include mineral oil, petroleum jelly, squalene and isoparaffins.

The iron chelator molecule may be present within a cream or lotion at a concentration of about 0.1 mM, about 1 mM, about 10 nM, about 100 mM, about 500 mM, about 1000 mM, or any value therebetween.

Films or patches. Another exemplary formulation for use in the methods described herein employs transdermal delivery devices (“patches”). Such transdermal patches may be used to provide continuous or discontinuous infusion of an iron chelator in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The iron chelator molecule may be present within a film or patch at a concentration of about 0.1 to about 10 mg/cm2, about 0.5 mg/cm2 to about 10 mg/cm2, about 0.5 mg/cm2 to about 7 mg/cm2, about 0.5 mg/cm2 to about 5 mg/cm2, about 1.0 mg/cm2 to about 10 mg/cm2, or about 1 mg/cm2 to about 10 mg/cm2. The dosage may depend on the nature of the iron chelator molecule, e.g., the number of iron moieties that may be chelated by one iron chelator molecule.

The total dose of the iron chelator molecule, such as deferoxamine, deferiprone, or deferasirox, provided in a patch or film may be at least about 500 mg, at least about 1.0 g, and not more than about 6.0 g, not more than about 5.0 g, or not more than about 2.0 g, and may be from about 1.0 g to about 3.0 g, e.g. about 100 mg.

The film or patch including an iron chelator molecule may include an extended release formulation and may therefore include any of the above mentioned components such as reverse micelles, stabilizers, permeability enhancers, other molecules which prevent crystallization, biodegradable polymers, backings, and the like as described above. A patch formulation may include an adhesive layer which may help to hold the formulation in contact with the skin to be treated. The patch formulation may, but need not be a film formulation but may be a cream formulation. Any suitable dermatologically appropriate adhesive may be used, as is known in the art, one nonlimiting example being an acrylic type adhesive. Also, the patch may have a backing, which may be an occlusive backing, such as a polyurethane backing to secure the formulation to the region being treated.

Plasticizer. A plasticizer may be, but is not required to be included in the formulations, particularly for a thin film or patch. Any suitable plasticizer may be used, including but not limited to chitosan, sorbitol, glycerol, polyethylene glycol 400, dibutyl sebacate, diethyl phthalate, vegetable oils, triacetin, acetylated monoglycerides. The plasticizer, if present, may be present in the formulation in a concentration of about 10%, about 20%, about 30% or about 40% w/w of the polymers. In some variations, di-n-Butyl phthalate may be used as a plasticizer at a concentration of about 30% weight-in-weight of polymers.

Kits for treating a subject are provided, including a container including a pharmaceutically acceptable composition including an iron-chelating compound, which may be any iron-chelating compound described herein, and instructions for its use. The pharmaceutically acceptable composition may be formulated as any formulation described herein for delivering an iron-chelating compound, and may be a lotion, a gel, a thin film or a transdermal patch. In some variations, the kit may include applicators for applying the pharmaceutically acceptable compositions to the affected region of the subject.

### EXAMPLE

Materials and Methods

Topical deferoxamine (also known as desferrioxamine, desferoxamine, DFO) is used in several concentrations depending on experimental conditions. Additionally, a number of other iron chelators as described herein such as deferiprone, deferasirox find use.

Transdermal Delivery of Iron Chelators. A patch is designed for transdermal delivery system, including an adhesive, impermeable backing membrane, and a release liner containing iron chelator (50-200 mg) dispersed or super-saturated within a biodegradable polymer. Preparation of transdermal patch includes a mixture of polymers (total weight, 400 mg, weighed in a 7:1 ratio of Ethyl Cellulose and Polyvinyl Pyrrolidone) and iron chelators, dissolved in 10 ml of chloroform. Additives are also included that prevent small molecule crystallization, resulting in enhanced drug release. Di-n-Butyl phthalate is then used as a plasticizer (30% weight-in-weight of polymers). To create the final release liner, this solution is then poured onto a sterile glass petri dish and dried at room temperature. The uniform dispersion, 2 ml each, is cast onto a 4% Polyvinyl Alcohol backing membrane and dried at 40 C for 6 hours. Finally, the backing membrane is attached to the contact adhesive (3M Tegaderm) keeping the matrix side upward. After 24 hours, the transdermal films are cut with a Delasco KP-16 mm circular punch biopsy and stored in a desiccator until further use.

Immunohistochemstry. CD31 staining is performed on paraffin embedded 5-micron wound sections (1:50, Santa Cruz Biotechnology, Santa Cruz, Calif.) diluted in blocking goat serum overnight at 4° C. Sections were then stained with goat anti-rat FITC secondary antibody (Santa Cruz Biotechnology, Santa Cruz, Calif.) for 1 hour at room temperature. Sections are then mounted with Vectashield plus DAPI (Vector Laboratories, Burlingame, Calif.), and analyzed using a Zeiss Axioplan 2 light-fluorescent microscope (Carl Zeiss Vision, Germany) equipped with Zeiss AxioCam HR digital imaging software (Carl Zeiss Vision). CD31+ vessel counts are performed by counting the number of capillaries present in 4 separate 40× high power fields (HPF). TUNEL (Roche) staining was also performed. All measurements are performed by two blinded observers.

Superoxide Assay (DHE). 30 μm fresh frozen sections are washed with PBS and stained with 10 μM Dihydroethidium (DHE, invitrogen) at 37 C for 30 minutes. Slides are then washed with PBS, and Vectashield with DAPI is added.

Western Blot. 50 μg of nuclear protein extract using a NE-PER kit (Pierce) and supplemented with protease inhibitor cocktail (company). Lysate protein concentrations are determined with the Micro BCA Protein Assay Kit (Pierce). Then 50 μg of nuclear lysate is fractionated by SDS-polyacrylamide gel electrophoresis (PAGE) and analyzed by immunoblotting. Protein detection is performed with primary antibodies against iron chelators (1:500 dilution, Novus Biologicals, Littleton, Colo.) and (3-actin (1:5000 dilution, Lab Vision, Fremont, Calif.) in 5%/TBS-T overnight at 4° C. Blots are then incubated with the corresponding HRP-linked secondary antibodies (1:10,000 dilution, BD Pharmingen, San Jose, Calif.) for one hour at room temperature. Blots are developed with ECL detection reagent (Amersham, UK) and exposed for 1-10 minutes using Biomax-MS film (Kodak, Rochester, N.Y.).

### Example 1

DFO formulations: film. Table 1 lists some of the formulations used in the present invention.

TABLE 1

| Formulation 1 | Formulation 2 | Formulation 3 |
|---|---|---|
| DFO 100 mg | DFO 100 mg | DFO 100 mg |
| Tween-80 100 mg | PEG 6000 100 mg | Tween-80 100 mg |
| Span-20 100 mg | Cetyl alcohol 30 mg | Span-20 100 mg |
| PVP (360k) 50 mg | Plurol Oleique 50 mg | PVP (10k) 50 mg |
| Cetyl alcohol 30 mg | Ethyl cellulose 500 mg | Cetyl alcohol 30 mg |
| Plurol Oleique 50 mg | | Plurol Oleique 50 mg |
| Ethyl cellulose 350 mg | | Ethyl cellulose 350 mg |
| Formulation 4 | Formulation 5 | Formulation 6 |
| DFO 100 mg | DFO 100 mg | DFO 100 mg |
| PVP (10k) 50 mg | PVP (10k) 80 mg | CTAB 130 mg |
| PEG-6000 100 mg | Cetyl alcohol 50 mg | PVP 120 mg |
| Cetyl alcohol 30 mg | Plurol Oleique 120 mg | Ethyl cellulose 400 mg |
| Plurol Oleique 50 mg | Ethyl cellulose 400 mg | |
| Ethyl cellulose 500 mg | | |

Preparation of DFO Patch (general procedure of all six formulations above): Weigh each of components for the amount as given in the formulation table. Dissolve all the components separately as follows: (e.g., Formulation 1 for 100 cm² patch), Ethyl Cellulose (ethoxy content: 48%, 110 cps supplied by Acros Organics, N.J.) in 5 mL of ethanol, Polyvinyl Pyrrolidone (MW: 10,000, Sigma, St Louis Mo.) in 1 ml of ethanol, DFO (deferoxamine mesylate) 1 mL of 50% ethanol-water mixture, Tween-80 and Span-20 in 1 mL of ethanol, Plurol Oleique in 1 mL of ethanol, cetyl alcohol in 1 mL of ethanol. All of the solutions were mixed, and made up to a total volume of 10 mL with ethanol. Stir the solution for 30 min. Pour the solution (10 mL) to a Teflon coated tray (100 cm²-10×10 cm) (or 0.1 mL per cm²). Evaporate the ethanol by drying it at 37° C. for 12 hours. The dried films are removed and cut to the required sizes. The patches are attached to the adhesive membrane (3M Tegaderm) and stored in desiccator (ready to use).

Table 2 describes various source of excipients used in the formulation.

TABLE 2

| | | |
|---|---|---|
| Ethyl cellulose, | variable | Thermo Fisher Scientific |
| ethoxyl content | | Geel-Belgium |
| 48%, 22 cps | | |
| Polyvinyl | average mol | Sigma-Aldrich, Inc. St Louis, MO |
| Pyrrolidone | wt 10,000 | |
| Absolute | 46.07 | Sigma-Aldrich, Inc. St Louis, MO |
| Ethanol | | |
| (200 Proof) | | |
| Deferoxamine | 656.8 | EMD Biosciences, Inc. La Jolla, CA |
| mesylate | | |
| Tween 80 | 1310 | MP Biomedicals, LLC Solon, OH |
| Span 20 | 346.47 | Sigma-Aldrich, Inc. St Louis, MO |
| Plurol Oleique | 726.93 | Pharmaceutical Division |
| | | Gattefossé USA |
| | | Plaza I, 115 West Century Road, Suite 340 |
| | | Paramus, NJ 07652 |

Another formulation used in the present invention includes:

Formulation 7

DFO 100 mg
PVP 80 mg
Cetyl alcohol 50 mg
Plurol Oleique 120 mg
Ethyl cellulose 400 mg
(Ethyl alcohol 10 ml)

Formulation 7 can be prepared as follows:

(1) Dissolve 1600 mg of ethyl cellulose in 24 ml of ethanol (stirring overnight, turbid solution).
(2) Combine Cetyl alcohol, PVP, Plurol Oleique, and 16 ml of ethanol; and stir.
(3) Weigh out 400 mg of DFO, wet it with about 400 μl of water, add solution from (2) and stir (will be a suspension).
(4) Combine (1) and (3), stir.
(5) Setup 8-well tray on a flat level surface at a temperature of 37° C.
(6) Dispense 4 ml of (4) to each well, cover with tissue/paper towel. Let dry overnight.
(7) Using spatula remove patches from the tray. Store in an airtight container at room temperature

Another formulation used in the present invention includes:

Formulation 8

DFO 100 mg
PVP 80 mg
Cetyl alcohol 50 mg
Plurol Oleique 120 mg
Ethyl cellulose 400 mg
(Ethyl formate 10 ml)

Formulation 8 can be prepared as follows:

(1) Dissolve 1600 mg of ethyl cellulose in 24 ml of ethyl formate (stirring overnight, turbid solution).
(2) Combine Cetyl alcohol, PVP, Plurol Oleique, and 16 ml of ethyl formate; and stir.
(3) Weigh out 400 mg of DFO, wet it with about 600 μl of water, add 600 μl of solution from
(2). Add rest of (2) and stir (will be a clear solution).
(4) Combine (1) and (3), and stir (will become cloudy, but will not sediment).
(5) Setup 8-well tray on a flat level surface at a temperature of 37° C.
(6) Dispense 4 ml of (4) to each well, cover with tissue/paper towel. Let dry overnight.
(7) Using spatula remove patches from the tray. Store in an airtight container at room temperature.

Another formulation used in the present invention includes:

Formulation 9

DFO 100 mg
PVP 80 mg
CTAB 80 mg
Ethyl cellulose 500 mg
(Ethyl alcohol 10 ml)

Formulation 9 can be prepared as follows:

(1) Dissolve 1600 mg of ethyl cellulose in 24 ml of ethyl alcohol (stirring overnight, turbid solution).
(2) Combine CTAB, PVP, and 16 ml of ethyl alcohol; and stir.
(3) Weigh out 400 mg of DFO, wet it with about 600 μl of water, add 600 μl of solution from (2). Add rest of (2) and stir (will be turbid solution with visible swirls when shaken).
(4) Add 1.2 ml of water, stir overnight. Add another 1 ml of water.
(5) Combine (1) and (4), and stir (will become cloudy, but will not sediment).
(6) Setup 8-well tray on a flat level surface at a temperature of 37° C.
(7) Dispense 4 ml of (5) to each well, cover with tissue/paper towel. Let dry overnight.
(8) Using spatula remove patches from the tray. Store in an airtight container at room temperature.

Another formulation used in the present invention includes

Formulation 10

DFO 100 mg
PVP 80 mg
CTAB 80 mg
Ethyl cellulose 400 mg
(Ethyl formate 10 ml)

Formulation 10 can be prepared as follows:

(1) Dissolve 1600 mg of ethyl cellulose in 24 ml of ethyl formate (stirring overnight, turbid solution).
(2) Combine CTAB, PVP, and 16 ml of ethyl formate; and stir (will not dissolve). Add 500 μl×3 (1.5 ml) of water (will be a turbid solution)
(3) Weigh out 400 mg of DFO, wet it with about 600 μl of water, add 600 μl of solution from (2). Add rest of (2) and stir (will be turbid solution with visible swirls when shaken).
(4) Add 1.2 ml of water, stir overnight. Add another 1 ml of water.
(5) Combine (1) and (4), and stir (will become cloudy, but will not sediment).
(6) Setup 8-well tray on a flat level surface at a temperature of 37° C.
(7) Dispense 4 ml of (5) to each well, cover with tissue/paper towel. Let dry overnight.
(8) Using spatula remove patches from the tray. Store in an airtight container at room temperature.

The patches using formulations 9 and 10 above can also be made by substituting a different solvent for ethyl formate (e.g., isooctane, n-heptane, a super critical fluid of CO2, and the like). These substitute solvents can be used with volumes up to 2 times that of what is described for ethyl formate above.

Other combinations of surfactants, stabilizers, matrix molecules and solubilizers may be used. Different iron chelators may be formulated in place of the DFO. When formulating a more hydrophobic iron chelator, a reverse micelle may not be employed (e.g., cetyl alcohol as used herein, or any other non-ionic surfactant may not form a reverse micelle) and the ratios of stabilizers such as PVP may vary greatly. Other surfactants may be used, other than non-ionic surfactants.

DFO delivery device. A transdermal delivery system was used to deliver DFO into the dermal tissue of the mice. The delivery system comprised a dry film comprising DFO at a concentration of 13.4% weight/weight % of film encapsulated in a reverse micelle with a non-ionic surfactant stabilized by polyvinylpyrrolidone (PVP) in an ethylcellulose matrix, cut into a ⅝ inch circle and covered by a silicon sheet of the same size.

### Example 2

DFO formulations: lotion or cream. Table 3 lists some of the formulations which may be used as creams or lotions.

TABLE 3

| Formulation 11 | Formulation 12 | Formulation 13 |
|---|---|---|
| DFO 100 mg | DFO 100 mg | DFO 100 mg |
| PVP (10k) 80 mg | PVP (10k) 80 mg | PVP (10k) 80 mg |
| Cetyl alcohol 25 mg | Cetyl alcohol 50 mg | Cetyl alcohol 100 mg |
| Plurol Oleique 60 mg | Plurol Oleique 120 mg | Plurol Oleique 240 mg |
| Cream base (Vaseline/Petroleum | Cream base (Vaseline/ | Cream base (Vaseline/ |
| white) 400 mg | Petroleum white) 400 mg | Petroleum white) 400 mg |
| Weight of surfactants: 12.8% | Weight of surfactants: 22.7% | Weight of surfactants: 40.7% |
| Total weight: 665 | Total weight: 750 | Total weight: 835 g |

Formulations 11, 12, and 13 can be prepared as in the following steps:

(1) Add 100 uL of water to the 100 mg of DFO.
(2) Add 0.9 mL ethanol in a two step addition. Add 0.4 mL, mix the components, and then add the remaining 0.5 mL.
(3) Add the selected amount of polyvinyl pyrrolidone (PVP) dissolved in 1 mL ethanol.
(4) Add the selected amount of Cetyl alcohol in 1 mL of ethanol.
(5) Add the selected amount of Plurol Oleique in 1 mL of ethanol.
(6) Mix the combined mixtures (from (2), (3), (4) and (5) for 10 minutes with sonication.
(7) In a separate container, melt 400 mg Vaseline by warming on a magnetic stirrer, with the container in a hot bath.
(8) Add the DFO-containing mixture from (6) and add slowly to the molten Vaseline while stirring.
(9) Stir for 30 minutes or until the ethanol evaporates.
(10) Cool down the mixture while stirring to obtain a homogeneous cream.

It can be understood that many variations may be made to the cream formulations 11, 12, 13. Vaseline can be replaced with Eucerin, eucerinum anhydricum, Bentonite, PEG, mulgafarin, and Decoderm basiscreme.

Other oil- or water-based materials may be substituted for the cream base in order to provide a lotion formulation, as described throughout the specification. Also, the amount of iron chelator may be different, and different iron chelators may be formulated, using these examples as a general guide.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

List of some embodiments of the disclosure.

1. A method of treating an aesthetic skin condition in a subject's skin, including: contacting the subject's skin in need thereof with an effective amount of an iron chelator compound or a pharmaceutically acceptable salt thereof, or a composition including an iron chelator compound or a pharmaceutically acceptable salt thereof, where the aesthetic skin condition is prevented or treated.

2. The method of embodiment 1, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

3. The method of embodiment 1 or 2, where the iron chelator compound is deferoxamine.

4. The method of any one of embodiments 1 to 3, where the aesthetic skin condition is a decreased hair density, a wrinkle, a fine line on skin, dry, flaky or itchy skin, alopecia, aging, skin discoloration, a sun spot, an age spot, a dark circle under eyes, bruising following injury or surgery, a liver spot, scar, spider vein, or rosacea.

5. The method of any one of embodiments 1 to 4, where the effective amount of an iron chelator compound or a pharmaceutically acceptable salt thereof, or a composition including an iron chelator compound or a pharmaceutically acceptable salt thereof is administered topically.

6. The method of any one of embodiments 1 to 5, where the iron chelator compound is encapsulated in a reverse micelle structure with at least one surfactant.

7. The method of embodiment 6, where contacting the subject's skin with the composition including the iron chelator compound further includes releasing the iron chelator compound from the composition over a treatment period, and penetrating the iron chelator compound into the skin in need of treatment.

8. The method of embodiment 7, where releasing the iron chelator compound further includes releasing the iron chelator compound from within a matrix of the composition.

9. The method of embodiment 8, where the matrix includes ethylcellulose.

10. The method of any of embodiments 1-9, where the composition further includes polyvinylpyrrolidone (PVP).

11. The method of any one of embodiments 1 to 10, where the iron chelator compound has a concentration of at least about 0.1% and not more than about 40% as weight/weight percent of the composition.

12. The method of any one of embodiments 1-11, where the composition is a cream or a lotion.

13. The method of any one of embodiments 1-12, where the composition is a film.

14. The method of embodiment 13, where applying includes applying to the skin a transdermal patch containing the composition.

15. The method of any one of embodiments 1 to 4, where the effective amount of an iron chelator compound or a pharmaceutically acceptable salt thereof, or a composition including an iron chelator compound or a pharmaceutically acceptable salt thereof is administered orally.

16. A formulation comprising an iron chelator compound or a pharmaceutically acceptable salt thereof in a cream, lotion or film composition formulated for transdermal aesthetic skin treatment.

17. The formulation of embodiment 16, wherein the iron chelator compound is stabilized in the cream, lotion or film composition.

18. The formulation of embodiment 16 or 17, wherein the iron chelator compound is encapsulated in a reverse micelle structure.

19. The formulation of embodiment 18, where the iron chelator compound or pharmaceutically acceptable salt thereof is encapsulated with at least one surfactant within the reverse micelle.

20. The formulation of embodiment 19, where the at least one surfactant includes one or more of TWEEN 85® (Polyoxyethylene (20) Sorbitan Trioleate); phospholipids; fatty acid esters; TRITON X-100® (Octylphenol ethylene oxide condensate); AOT (dioctyl sulfosuccinate)-TWEEN 80® (Polysorbate 80); Span20 (sorbitan monolaurate); AOT-DOLPA (dioleyl phosphoric acid); AOT-OPE4 (p,t-octylphenoxyethoxyethanol); CTAB (cetyl trimethylammonium bromide)-TRPO (mixed trialkyl phosphine oxides); fatty alcohols; and CTAB (cetyl trimethylammonium bromide).

21. The formulation of embodiment 19 or 20, where the at least one surfactant includes at least one of Polysorbate 80 and sorbitan monolaurate (Span20).

22. The formulation of any one of embodiments 18 to 21, where the at least one surfactant is present at a concentration of 1% w/w to 25% w/w.

23. The formulation of any one of embodiments 16 to 22, where the formulation further includes polyvinylpyrrolidone.

24. The formulation of embodiment 23, where the polyvinylpyrrolidone is present at a concentration of 0.1% to 25% w/w.

25. The formulation of any one of embodiments 16 to 24, where the iron-chelating compound is present within the formulation in a concentration from 1% to 35% w/w.

26. The formulation of embodiment 25, where the iron-chelating compound is present within the formulation at a concentration of 13% w/w.

27. The formulation of any one of embodiments 16 to 26, where the formulation is a lotion or gel.

28. The formulation of any one of embodiments 16 to 26, further including a matrix.

29. The formulation of 28, where the matrix is a biodegradable polymer.

30. The formulation of embodiment 29, where the biodegradable polymer includes ethyl cellulose.

31. The formulation of embodiment 30, where ethyl cellulose is present at a concentration from 25% w/w to 75% w/w.

32. The formulation of any one of embodiments 16 to 31, where the formulation of the iron-chelating compound is a film or a patch.

33. The formulation of any one of embodiments 16 to 32, where the formulation is configured to be compatible with facial skin or a scalp of a subject.

34. A method of treating a skin condition associated with oxidation of skin cells, the method including topically applying to skin in need thereof an iron chelator compound or a pharmaceutically acceptable salt thereof, or a composition including an iron chelator compound, or a pharmaceutically acceptable salt thereof, where topical application of the compound, salt or composition treats oxidation of skin cells.

35. The method of embodiment 34, where applying includes applying to the skin a transdermal patch containing the composition.

36. The method of embodiment 34 or 35, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

37. The method of any one of embodiments 34 to 36, where the iron chelator compound is DFO.

38. The method of any one of embodiments 34 to 37, where the iron chelator compound is encapsulated in a reverse micelle structure with a surfactant.

39. The method of embodiment 38, where the iron chelator compound is encapsulated within the reverse micelle structure within a matrix.

40. The method of embodiment 39, where the matrix includes a biodegradable polymer.

41. The method of any one of embodiments 34 to 40, where the composition further includes polyvinylpyrrolidone (PVP).

42. A method for preventing and/or treating an aesthetic skin condition in a subject's skin, the method including: contacting the subject's skin in need thereof with an effective amount of an iron chelator compound or a pharmaceutically acceptable salt thereof, or a composition including an iron chelator compound or a pharmaceutically acceptable salt thereof, where the aesthetic skin condition is prevented or treated.

43. The method of embodiment 42, where the aesthetic skin condition is a decreased hair density, skin thinning a wrinkle, a fine line on skin, alopecia, aging, skin discoloration, a sun spot, an age spot, a dark circle under eyes, bruising following injury or surgery, a liver spot, scar, spider vein, or rosacea.

44. The method of embodiment 42 or 43, where the iron chelator compound is encapsulated in a reverse micelle structure with a surfactant within a matrix.

45. The method of embodiment 44, where the encapsulated iron chelator compound is released from the matrix over a treatment period, and penetrated into the skin in need of treatment.

46. The method of any one of embodiments 42-45, where the iron chelator compound is formulated into a composition suitable for transdermal delivery.

47. The method of embodiment 46, where the composition is a film.

48. The method of embodiment 46 or 47, where the iron chelator compound is contained in a patch.

49. The method of any one of embodiments 42-48, where the iron chelator compound is formulated into a composition suitable for topical delivery.

50. The method of embodiment 49, where the composition is a cream or lotion.

51. The method of any one of embodiments 42-48, where the contacting includes applying to the skin with a transdermal patch including a film including an iron chelator compound.

52. The method of any one of embodiments 42-51, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

53. The method of any one of embodiments 42-52, where the iron chelator compound is deferoxamine (DFO).

54. The method of any one of embodiments 42-53, where the matrix includes ethylcellulose.

55. The method of any one of embodiments 42-54, where the composition further includes polyvinylpyrrolidone (PVP).

56. The method of any one of embodiments 42-55, where the iron chelator compound has a concentration of at least about 0.1% and not more than about 40% as weight/weight percent of the composition.

57. The method of embodiment 47, where the iron chelator compound has a concentration of at least about 0.1% and not more than about 40% as weight/weight percent of the film.

58. A method for improving, preventing and/or treating an aesthetic skin condition in a subject, the method including: administering to the subject in need thereof an effective amount of an iron chelator compound, or a pharmaceutically acceptable salt thereof, or a composition containing an iron chelator compound or a pharmaceutically acceptable salt thereof, thereby improving and/or treating the aesthetic skin condition.

59. The method of embodiment 58, where the iron chelator compound is formulated into a composition suitable for oral administration.

60. A method for preventing and/or treating an aesthetic skin condition in a subject's skin, the method including: contacting the subject's skin in need thereof with a transdermal patch including a film including an iron chelator compound or a pharmaceutically acceptable salt thereof encapsulated in a reverse micelle structure with surfactant within a matrix, where the encapsulated iron chelator compound is released from the matrix over a treatment period, and penetrated into the skin.

61. The method of embodiment 60, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

62. The method of embodiment 60 or 61, where the iron chelator compound is DFO.

63. A method of lightening skin or evening skin tone, the method including topically applying to skin in need thereof an iron chelator compound or a pharmaceutically acceptable salt thereof, or a composition including an iron chelator compound, where topical application of the compound, salt or composition lightens skin or evens skin tone.

64. The method of embodiment 63, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

65. The method of embodiment 63 or 64, where the iron chelator compound is DFO.

65. The method of any one of embodiments 63-65, where applying includes applying to the skin a transdermal patch containing the composition.

67. The method of any one of embodiments 63-66, where the iron chelator compound is encapsulated in a reverse micelle with a surfactant within a matrix.

68. The method of any one of embodiments 63 to 67, where the compositions further includes polyvinylpyrrolidone (PVP)

69. A method of reducing the appearance of symptoms associated with erythema, the method including topically applying to skin in need thereof an iron chelator compound, or a pharmaceutically acceptable salt thereof, or a composition including an iron chelator compound, or a pharmaceutically acceptable salt thereof, where topical application of the compound, salt or composition reduces the appearance of symptoms associated with erythema.

70. The method of embodiment 69, where the applying includes applying to the skin a transdermal patch containing the composition.

71. The method of embodiment 69 or 70, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

72. The method of any one of embodiments 69 to 71, where the iron chelator compound is DFO.

73. The method of any one of embodiments 69 to 72, where the iron chelator compound is encapsulated in a micelle structure with a surfactant within a matrix.

74. The method of any one of embodiments 69 to 73, where the composition further includes polyvinylpyrrolidone (PVP).

75. A method of treating dry, flaky, or itchy skin or reducing the appearance of uneven skin tone, the method including topically applying to skin in need thereof an iron chelator compound or a pharmaceutically acceptable salt thereof, or a composition including an iron chelator compound or a pharmaceutically acceptable salt thereof, where topical application of the compound, salt or composition treats dry, flaky, or itchy skin or reducing the appearance of the uneven skin.

76. The method of embodiment 75, where the applying includes applying to the skin a transdermal patch containing the composition.

77. The method of embodiment 75 or 76, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

78. The method of any one of embodiments 75 to 77, where the iron chelator compound is DFO.

79. The method of any one of embodiments 75 to 78, where the iron chelator compound is encapsulated in a micelle structure with a surfactant within a matrix.

80. The method of any one of embodiments 75 to 79, where the composition further includes polyvinylpyrrolidone (PVP).

81. A method of reducing the appearance of fine lines or wrinkles of skin, the method including topically applying to the skin in need thereof an iron chelator compound, or a pharmaceutically acceptable salt thereof, or a composition including an iron chelator compound, or a pharmaceutically acceptable salt thereof, where topical application of the composition reduces the fine lines or wrinkles of the skin.

82. The method of embodiment 81, where the applying includes applying to the skin a transdermal patch containing the composition.

83. The method of embodiment 81 or 82, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

84. The method of any one of embodiments 81 to 83, where the iron chelator compound is DFO.

85. The method of any one of embodiments 81 to 84, where the iron chelator compound is encapsulated in a micelle structure with a surfactant within a matrix.

86. The method of any one of embodiments 81 to 85, where the composition further includes polyvinylpyrrolidone (PVP).

87. A method of treating hyperpigmentation of skin, the method including topically applying to the skin in need thereof an iron chelator compound, or a pharmaceutically acceptable salt thereof, or a composition including an iron chelator compound or a pharmaceutically acceptable salt thereof, where topical application of the compound, salt, or composition treats hyperpigmentation of the skin.

88. The method of embodiment 87, where the applying includes applying to the skin a transdermal patch containing the composition.

89. The method of embodiment 87 or 88, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

90. The method of any one of embodiments 87 to 89, where the iron chelator compound is DFO.

91. The method of any one of embodiments 87 to 90, where the iron chelator compound is encapsulated in a micelle structure with a surfactant within a matrix.

92. The method of any one of embodiments 87 to 91, where the composition further includes polyvinylpyrrolidone (PVP).

93. A method of thickening hair or treating or preventing hair loss on the scalp, the method including topically applying to the skin in need thereof an iron chelator compound, or a pharmaceutically acceptable salt thereof, or a composition including an iron chelator compound or a pharmaceutically acceptable salt thereof, where topical application of the compound, salt or composition thickens hair or treats or prevents hair loss on the scalp.

94. The method of embodiment 93, where the applying includes applying to the skin a transdermal patch containing the composition.

95. The method of embodiment 93 or 94, where the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

96. The method of any one of embodiments 93 to 95, where the iron chelator compound is DFO.

97. The method of any one of embodiments 93 to 96 where the iron chelator compound is encapsulated in a micelle structure with a surfactant within a matrix.

98. The method of any one of embodiments 93 to 97, where the composition further includes polyvinylpyrrolidone (PVP).

99. A kit for treating aesthetic skin conditions, including a composition comprising an iron chelator compound; and directions for its use.

100. The kit of embodiment 99, where the composition is the formulation of any one of embodiments 16 to 33.

101. The kit of embodiments 99 or 100, wherein the composition further includes a moisturizer, emollient, or dermatologically acceptable vehicle.

102. The kit of any one of embodiments 99 to 101, where the composition is a lotion or a cream.

103. The kit of any one of embodiments 99 to 102, where the composition is a film or a patch.

104. The kit of embodiment 99 to 103, where the kit further includes an applicator for the composition.

What is claimed is:

1. A method of treating an aesthetic skin condition in a subject's skin, the aesthetic skin condition selected from a group consisting of decreased hair density, thinning skin, a wrinkle, a fine line on skin, dry, flaky or itchy skin, alopecia, aging, skin discoloration, a sun spot, an age spot, a dark circle under eyes, bruising following injury or surgery, a liver spot, scar, and spider vein, the method comprising:
contacting the subject's skin in need thereof with an effective amount of an iron chelator compound or a pharmaceutically acceptable salt thereof, or a composition comprising an iron chelator compound or a pharmaceutically acceptable salt thereof, wherein the aesthetic skin condition is prevented or treated.

2. The method of claim 1, wherein the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

3. The method of claim 1, wherein the iron chelator compound is deferoxamine.

4. (canceled)

5. The method of claim 1, wherein the effective amount of an iron chelator compound or a pharmaceutically acceptable salt thereof, or a composition comprising an iron chelator compound or a pharmaceutically acceptable salt thereof is administered topically.

6. The method of claim 1, wherein the iron chelator compound is encapsulated in a reverse micelle structure with at least one surfactant.

7. The method of claim 6, wherein contacting the subject's skin with the composition comprising the iron chelator compound further comprises releasing the iron chelator compound from the composition over a treatment period, and penetrating the iron chelator compound into the skin in need of treatment.

8. The method of claim 7, wherein releasing the iron chelator compound further comprises releasing the iron chelator compound from within a matrix of the composition.

9. (canceled)

10. (canceled)

11. The method of claim 1, wherein the iron chelator compound has a concentration of at least about 0.1% and not more than about 40% as weight/weight percent of the composition.

12. The method of claim 1, wherein the composition is a cream or a lotion.

13. The method of claim 1, wherein the composition is a film.

14. The method of claim 13, wherein applying comprises applying to the skin a transdermal patch containing the composition.

15. (canceled)

16. A formulation comprising an iron chelator compound or a pharmaceutically acceptable salt thereof in a cream, lotion or film composition formulated for transdermal aesthetic skin treatment of decreased hair density, thinning skin, a wrinkle, a fine line on skin, dry, flaky or itchy skin, alopecia, aging, skin discoloration, a sun spot, an age spot, a dark circle under eyes, bruising following injury or surgery, a liver spot, scar, spider vein.

17. The formulation of claim 16, wherein the iron chelator compound is stabilized in the cream, lotion or film composition.

18. The formulation of claim 16, wherein the iron chelator compound is encapsulated in a reverse micelle structure.

19. The formulation of claim 18, wherein the iron chelator compound or pharmaceutically acceptable salt thereof is encapsulated with at least one surfactant within the reverse micelle.

20. (canceled)

21. (canceled)

22. (canceled)

23. (canceled)

24. (canceled)

25. The formulation of claim 16, wherein the iron-chelating compound is present within the formulation in a concentration from 1% to 35% w/w.

26. (canceled)

27. (canceled)

28. (canceled)

29. (canceled)

30. (canceled)

31. (canceled)

32. (canceled)

33. The formulation of claim 16, wherein the formulation is configured to be compatible with facial skin or a scalp of a subject.

34. A method of treating a skin condition associated with oxidation of skin cells, the method comprising topically applying to skin in need thereof an iron chelator compound or a pharmaceutically acceptable salt thereof, or a composition comprising an iron chelator compound, or a pharmaceutically acceptable salt thereof, wherein topical application of the compound, salt or composition treats oxidation of skin cells.

35. The method of claim 34, wherein applying comprises applying to the skin a transdermal patch containing the composition.

36. The method of claim 34, wherein the iron chelator compound is selected from deferoxamine, deferiprone, deferasirox, N,N′-Di(2-hydroxybenzyl)ethylenediamine-N,N′-diacetic acid monohydrochloride (HBED), pyridoxal isonicotinoyl hydrazine (PIH), desferrithiocin, and deferitrin.

37. (canceled)

38. (canceled)

39. (canceled)

40. (canceled)

41. (canceled)
